(19) United States
(12) Patent Application Publication
(10) Pub. No.: US 2023/0028847 A1
(43) Pub. Date: Jan. 26, 2023

(54) VAPORIZER DEVICE

(71) Applicant: JUUL Labs, Inc., San Francisco, CA (US)

(72) Inventors: Namhey Lee, Hayward, CA (US); Jia Li, Burlingame, CA (US); Maurizio Tarsia, San Carlos, CA (US)

(21) Appl. No.: 17/956,652

(22) Filed: Sep. 29, 2022

Related U.S. Application Data
(63) Continuation of application No. PCT/US21/25469, filed on Apr. 1, 2021.
(60) Provisional application No. 63/003,631, filed on Apr. 1, 2020.

(51) Int. Cl. A24F 40/30; A24F 40/05; A24F 40/57; A24F 7/00; B01F 23/10; B01F 25/433; B01F 25/431; B05B 17/00; B05B 7/16; B05B 7/00; B05B 7/24

(52) U.S. Cl. CPC A24F 40/30; A24F 40/05; A24F 40/57; A24F 7/00; B01F 23/12; B01F 25/4338; B01F 25/4331; B01F 25/431971; B05B 17/0646; B05B 7/1686; B05B 7/0012; B05B 7/2472

(57) ABSTRACT

A vaporizer device may include a vaporizer body configured to couple with at least one vaporizer cartridge including a first reservoir and a second reservoir. The vaporizer device may include a first aerosol generation mechanism configured to generate a first part of an aerosol by at least vaporizing a first aerosol component present in a first vaporizable material in the first reservoir. The vaporizer device may also include a second aerosol generation mechanism configured to generate a second part of the aerosol by vaporizing a first aerosol component present in a second vaporizable material at a second temperature. The first part of the aerosol and the second part of the aerosol may be delivered, via a mouthpiece, to a user of the vaporizer device.

### CROSS REFERENCE TO RELATED APPLICATION

This application claims priority to U.S. Provisional Application No. 63/003,631, entitled “VAPORIZER DEVICE” and filed on Apr. 1, 2020, the disclosure of which is incorporated herein by reference in its entirety.

### TECHNICAL FIELD

The subject matter described herein relates generally to vaporizers and, more specifically, to the vaporization of various ingredients in a vaporizable material.

### BACKGROUND

A vaporizer device may be used to generate an inhalable aerosol or a vapor. The aerosol may be generated by vaporizing a vaporizable material including a combination of ingredients such as, for example, one or more active ingredients (e.g., nicotine), flavorants (e.g., an ingredient to produce a flavored vapor), solvents (e.g., propylene glycol, glycerin), and/or the like. The vaporizable material may be in any form that is compatible with the vaporizer device including, for example, liquid, gel, solid, and/or the like.

### SUMMARY

Various aspects of the current subject matter address challenges associated with vaporizing a vaporizable material. Aspects of the current subject matter relate to apparatuses, methods, and systems for generating an aerosol that includes vaporizing at least a first ingredient of a first vaporizable material separately from a second ingredient of a second vaporizable material. Challenges associated with vaporizing a vaporizable material, particularly when the ingredients of the vaporizable material exhibit different levels of tolerance to heat, may be addressed by the inclusion of one or more of the features described herein or comparable/equivalent approaches as would be understood by one of ordinary skill in the art.

It will be understood from the context of this disclosure that the term “vaporize” is used to refer to creation of an aerosol, optionally by heating one or more aerosol components to a temperature sufficient to cause them to become gaseous and presenting conditions sufficient to cause at least some of the gas-phase one or more aerosol components to re-condense to form particles in an air stream that can be inhaled. The term vaporize is also used in this disclosure to refer to creation of aerosol particles via a process in which a transition to the gas phase is not a required intermediate step. For example, mechanical vaporizers such as piezo-electric vibration devices are discussed herein in terms of vaporizing one or more aerosol components even though such devices generally need not heat the one or more aerosol components to or above a temperature sufficient to cause the one or more aerosol components to become gaseous.

In one aspect, a vaporizer device includes a first reservoir and a second reservoir. The first reservoir contains or is configured to contain a first vaporizable material, and the second reservoir contains or is configured to contain a second vaporizable material. The vaporizer device also includes a first aerosol generation mechanism configured to create a first part of an aerosol that includes the at least one first aerosol component (e.g., one or more compounds or mixtures), wherein the first part of the aerosol is formed while the first vaporizable material is at a first temperature below a first vaporization temperature of the at least one first aerosol component, and a second aerosol generation mechanism configured to vaporize the second aerosol component (e.g., one or more compounds or mixtures) of the second vaporizable material to generate a second part of the aerosol. The first aerosol generation mechanism may optionally be configured to create a first part of aerosol that includes the at least one first aerosol component in a manner that does not heat the first vaporizable material to a first temperature that exceeds a vaporization temperature of the at least one first aerosol component. The second aerosol generation mechanism may optionally be configured to vaporize the second aerosol component in any manner, including heating the second vaporizable material to at least a second temperature that exceeds a vaporization temperature of the second aerosol component.

In another aspect, a vaporizer device includes a first reservoir and a second reservoir The first reservoir includes a first vaporizable material, which includes at least one first aerosol component, and the second reservoir includes a second vaporizable material, which includes at least one second aerosol component. The device also includes a first aerosol generation mechanism configured to vaporize the at least one first aerosol component to generate a first part of an aerosol and a second aerosol generation mechanism configured to vaporize at least one second aerosol component of the second vaporizable material to generate a second part of the aerosol. The first aerosol generation mechanism is configured to generate the first aerosol component without causing delivery of significant amounts of heat to the first vaporizable material and/or to the first aerosol component. The second aerosol generation mechanism is configured to vaporize, at a second temperature which can be near or above a vaporization temperature of the at least one second aerosol component to generate the second part of the aerosol.

In some variations, one or more of the following features may optionally be included in any feasible combination.

The vaporizer device may include a vaporizer device body that is configured to be re-usable and to therefore be joinable with at least one separable cartridge that includes certain parts of the vaporizer device. In some optional variations, the at least one separable cartridge may include the first and second reservoirs and at least part of each of the first and second aerosol generation mechanisms. In addition or alternatively, the at least one separable cartridge may include one or more of the first reservoir, the second reservoir, a mouthpiece, electrical connections for receiving power from a battery or other power source in the vaporizer device body when the vaporizer device body and cartridge are coupled.

The vaporizer device may optionally further include: a pressure sensor configured to detect a pressure change corresponding to an airflow through an air inlet in the vaporizer body; and a controller configured to respond to the pressure change by at least activating the first aerosol generation mechanism and/or the second aerosol generation mechanism.

In some variations, the controller may be further configured to control a first vaporization rate of the first vaporizable material by at least adjusting an amplitude, a frequency, and/or a duty cycle of a vibration associated with the first aerosol generation mechanism.

In some variations, the controller may be further configured to control a second vaporization rate of the second vaporizable material by at least adjusting a power level, a duty cycle of a power supply, and/or a target temperature associated with the second aerosol generation mechanism.

In some variations, the first temperature may be an ambient temperature.

In some variations, the first temperature may be lower than the second temperature.

In some variations, the second aerosol generation mechanism may include a heating element. The heating element may be configured to generate heat for heating the second vaporizable material to the second temperature in order to vaporize the second vaporizable material.

The first mechanism may optionally be a mechanical aerosol generator or an aerosol generator that uses one or more mechanical processes to create aerosol particles or droplets without requiring that the at least one first aerosol component is converted to the gas phase for subsequent recondensation.

In some variations, the first aerosol generation mechanism may include a piezoelectric actuator configured to generate an ultrasonic vibration. The ultrasonic vibration may vibrate a mesh screen to vaporize the first vaporizable material without generating heat to change the first temperature of the first vaporizable material.

In some variations, the piezoelectric actuator may be included in the first vaporizer cartridge.

In some variations, the first aerosol generation mechanism may include a mechanical horn configured generate an ultrasonic vibration. The ultrasonic vibration may vibrate a mesh screen to vaporize the first vaporizable material without generating heat to change the first temperature of the first vaporizable material.

In some variations, the mechanical horn may be included in the vaporizer body.

In some variations, the vaporizer body may be configured to couple with the first vaporizer cartridge including the first reservoir and the second reservoir.

In some variations, the vaporizer body may be configured to couple with the first vaporizer cartridge including the first reservoir and a second vaporizer cartridge including the second reservoir.

In some variations, the vaporizer device may further include: a mouthpiece configured to deliver, to a user, the first aerosol and the second aerosol, the mouthpiece including an aerosol outlet through which the first aerosol and the second aerosol exit the mouthpiece.

In some variations, the first aerosol may enter the mouthpiece through a first inlet in the mouthpiece and the second aerosol may enter the mouthpiece through a second inlet of the mouthpiece such that the first aerosol and the second aerosol enter the mouthpiece without any mixing.

In some variations, the first aerosol and the second aerosol may be mixed prior to entering the mouthpiece.

In some variations, the mouthpiece may be in fluid communication with a mixing chamber having one or more features configured to combine the first aerosol with the second aerosol.

In some variations, the one or more features may include a non-linear flow path and/or an obstacle.

In another aspect, a method includes: activating a first aerosol generation mechanism to vaporize at least one first aerosol component present in a first vaporizable material included in a first reservoir. The first aerosol generation mechanism is configured to vaporize the at least one first aerosol component without generating heat to change a first temperature of the first vaporizable material; activating a second mechanism to vaporize a second vaporizable material included in a second reservoir, the second mechanism configured to vaporize the second vaporizable material at a second temperature; and delivering, to a user, a first aerosol generated by vaporizing the first vaporizable material and a second aerosol generated by vaporizing the second vaporizable material.

In some variations, one or more of the following features may optionally be included in any feasible combination. The method may further include: detecting a pressure change corresponding to an airflow through an air inlet in the vaporizer body; and activating the first mechanism and/or the second mechanism in response to detecting the pressure change.

In some variations, the method may further include: controlling a first vaporization rate of the first vaporizable material by at least adjusting an amplitude, a frequency, and/or a duty cycle of a vibration associated with the first mechanism.

In some variations, the method may further include: controlling a second vaporization rate of the second vaporizable material by at least adjusting a power level, a duty cycle of a power supply, and/or a target temperature associated with the second mechanism.

In some variations, the first temperature may be an ambient temperature.

In some variations, the first temperature may be lower than the second temperature.

In some variations, the first mechanism may include a piezoelectric actuator and/or a mechanical horn configured to generate an ultrasonic vibration. The ultrasonic vibration may be configured to vaporize the first vaporizable material without generating heat to change the first temperature of the first vaporizable material.

In some variations, the ultrasonic vibration may vaporize the first vaporizable material by at least vibrating a mesh screen including a plurality of apertures.

In some variations, the second mechanism may include a heating element. The heating element may be configured to generate heat for heating the second vaporizable material to the second temperature in order to vaporize the second vaporizable material.

In some variations, the method may further include: coupling, with a vaporizer body of a vaporizer device, a vaporizer cartridge including the first reservoir and the second reservoir.

In some variations, the method may further include: coupling, with a vaporizer body of a vaporizer device, a first vaporizer cartridge including the first reservoir and a second vaporizer cartridge including the second reservoir.

In some variations, the first part of the aerosol and the second part of the aerosol may be delivered via a mouthpiece.

In some variations, the first aerosol may enter the mouthpiece through a first inlet in the mouthpiece and the second aerosol may enter the mouthpiece through a second inlet of the mouthpiece such that the first part of the aerosol and the second part of the aerosol enter the mouthpiece without any mixing.

In some variations, the first part of the aerosol and the second part of the aerosol may be mixed in a mixing chamber prior to entering the mouthpiece. The mixing chamber may be in fluid communication with the mouthpiece. The mixing chamber may include one or more features configured to combine the first aerosol with the second aerosol.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a top view of an example of a vaporizer device;

FIG. 2A depicts a block diagram illustrating an example of a vaporizer device, in accordance with some example embodiments;

FIG. 2B depicts a top view of an example of a mesh screen, in accordance with some example embodiments;

FIG. 2C depicts a perspective view of an example of a reservoir including a mesh screen, in accordance with some example embodiments;

FIG. 3 depicts a block diagram illustrating another example of a vaporizer device, in accordance with some example embodiments;

FIG. 4 depicts a block diagram illustrating another example of a vaporizer device, in accordance with some example embodiments;

FIG. 5 depicts a block diagram illustrating another example of a vaporizer device, in accordance with some example embodiments;

FIG. 6 depicts schematic diagrams illustrating examples of mechanisms for mixing aerosols in a vaporizer device with multiple reservoirs, in accordance with some example embodiments;

FIG. 7A depicts example configurations of a mouthpiece of a vaporizer device, in accordance with some example embodiments;

FIG. 7B depicts additional example configurations of a mouthpiece of a vaporizer device, in accordance with some example embodiments;

FIG. 8 depicts a block diagram illustrating an example of a computing system, in accordance with some example embodiments; and

FIG. 9 depicts a flowchart illustrating an example of a process for providing an aerosol, in accordance with some example embodiments.

When practical, similar reference numbers denote similar structures, features, or elements.

### DETAILED DESCRIPTION

A vaporizer device may include a cartridge containing a vaporizable material including multiple ingredients. Other types of vaporizer devices do not use separable cartridges such that the various components are included within a single device body. Such vaporizers may optionally have one or more re-fillable or non-refillable chambers or reservoirs for containing one or more vaporizable materials. A conventional vaporizer device, for example, may vaporize at least one component of the vaporizable material by heating the vaporizable material to cause the at least one aerosol component to be converted in the gas phase in a sufficiently high concentration to cause subsequent re-condensation of some or possibly all of the released at least one aerosol component to form aerosol particles. In such an approach, that every ingredient included in the vaporizable material is subject to a same high temperature despite having different chemical properties. For instance, the vaporizable material may include oil-based ingredients as well as water-based ingredients. With a conventional vaporizer device, the oil-based ingredients and the water-based ingredients may be heated collectively and thus subjected to the same temperature even when different chemical properties render, for example, the oil-based ingredients more (or less) tolerant of high temperatures than the water-based ingredients.

Vaporizing a combination of different ingredients collectively may present a variety of challenges and disadvantages. For example, different ingredients may react differently to heating and/or cooling. In particular, exposing some ingredients to heat may cause degradation whereas other ingredients may remain stable. Subjecting every ingredient in the vaporizable material to the same temperature may therefore compromise the flavor of the resulting aerosol. Additionally, vaporizing multiple ingredients collectively may prevent a user of the vaporizer device from controlling the rate of vaporizing each ingredient.

Accordingly, there is a need for a vaporizer device that may be configured to vaporize at least a first ingredient (also referred to as a first aerosol component) of a vaporizable material separately from a second ingredient (also referred to as a first aerosol component) of the vaporizable material. Such an approach may provide a variety of advantages including, for example, an ability to control various physical properties of the aerosol (e.g., the droplet size and/or the like) generated by the vaporizer device. For example, a vaporizable material may include multiple ingredients, each of which referred to herein a separate vaporizable material such as a first vaporizable material and a second vaporizable material. According to some implementations of the current subject matter, the first vaporizable material may be vaporized separately from the second vaporizable material such that the first vaporizable material may be subject to a different temperature than the second vaporizable material. For instance, at least one first aerosol component present in the first vaporizable material may be vaporized by a first mechanism that generates a first aerosol without substantially heating the first vaporizable material, for example through a piezo-electric or other mechanical droplet formation mechanism. The second vaporizable material may be vaporized by a second mechanism that generates a second aerosol by heating the second vaporizable material, for example by supplying heat to elevate the second vaporizable material to a temperature at or above a vaporization temperature of at least one second aerosol component present in the second vaporizable material. In doing so, a first part of a formed aerosol may include the at least one first aerosol component in a first droplet size (or size distribution) while a second part of the formed aerosol may include the at least one second aerosol component in a second droplet size (or size distribution). The vaporizer device may therefore be capable of delivering, in different quantities, at different vaporization rates, and/or to different areas of a respiratory tract of a user of the vaporizer device, the at least one first aerosol component and the at least one second aerosol component.

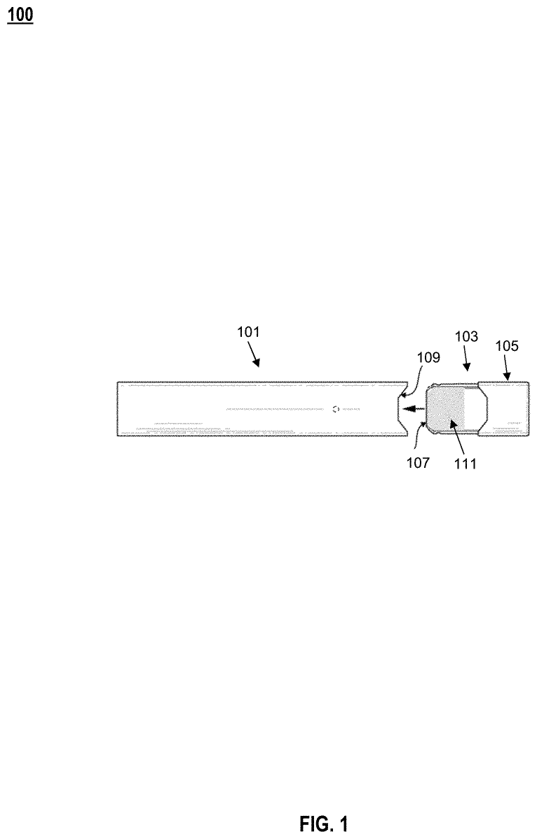

FIG. 1 depicts a top view of an example of a vaporizer device 100. Referring to FIG. 1, the vaporizer device 100 may include a vaporizer body 101 and a cartridge 103. As shown in FIG. 1, the vaporizer cartridge 103 may include a mouthpiece 105. An end section 107 of the cartridge 103 may be inserted into a receiving port 109 of the vaporizer body 101. The cartridge 103 may include a vaporizable material 111 including one or more ingredients such as, for example, active ingredients (e.g., nicotine and/or the like), flavorants (e.g., an ingredient to produce a flavored vapor), solvents (e.g., propylene glycol, glycerin, and/or the like), and/or the like.

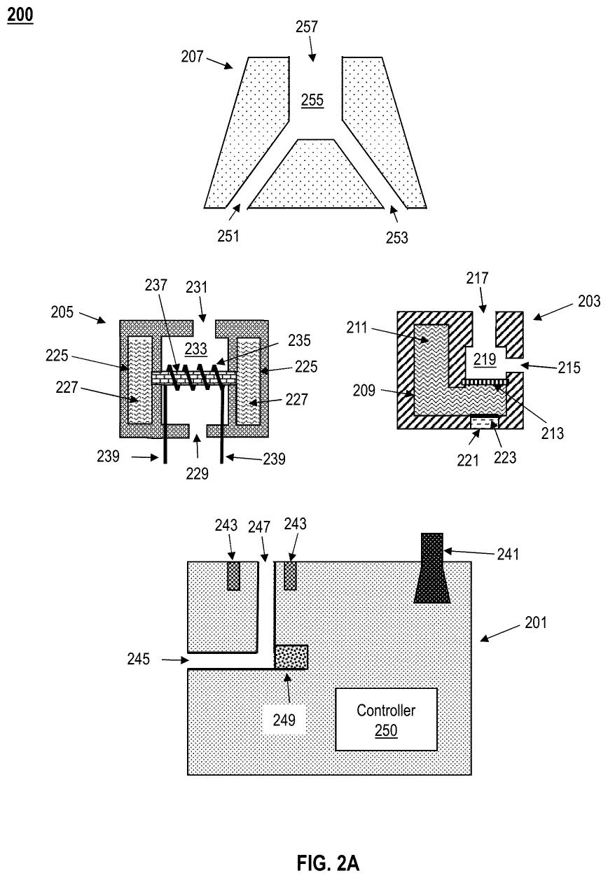

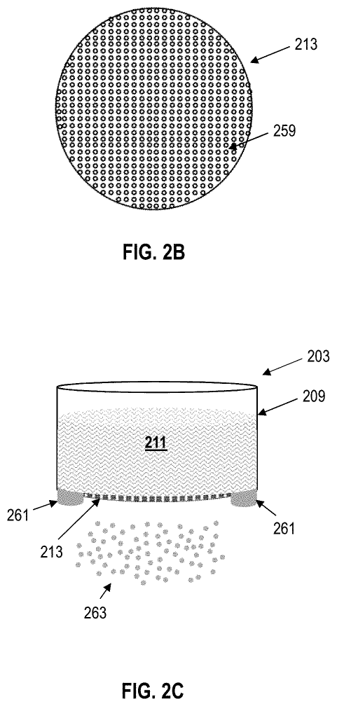

FIG. 2A depicts a block diagram illustrating example components of a vaporizer device 200, in accordance with some example embodiments. Referring to FIG. 2A, the vaporizer device 200 may include a vaporizer body 201, which may be configured to couple with the cartridge 103 including the vaporizable material 111. In some implementations of the current subject matter, the vaporizer device 200 shown in FIG. 2A, may include a first reservoir 203 holding a first vaporizable material 211 and a second reservoir 205 holding a second vaporizable material 227. The first vaporizable material 211 may include at least a first ingredient from the vaporizable material 111 while the second vaporizable material 227 may include a second ingredient from the vaporizable material 111. Instead of vaporizing every ingredient in the vaporizable material 111 collectively, the vaporizer device 200 may be configured to vaporize each of the first vaporizable material 211 and the second vaporizable material 227 individually. The resulting vapors may be delivered, as an inhalable aerosol through a mouthpiece 207 coupled with the reservoirs 203 and 205, with or without being combined.

As noted, the first reservoir 203 may include the first vaporizable material 211 and second reservoir 205 may include the second vaporizable material 227. Each of the first vaporizable material 211 and the second vaporizable material 227 may be in the form of a liquid, a solid, a gel, and/or the like. In some implementations of the current subject matter, the first reservoir 203 may include a content chamber 209 containing the first vaporizable material 211 (e.g., a flavorant), a mesh screen 213 having a plurality of apertures, an air inlet 215, an outlet 217, a vapor-air chamber 219, and an elastic pouch 221 containing a liquid 223 (e.g., water, a non-volatile oil, and/or the like) that may be different and separated from the first vaporizable material 211. In some example embodiments, the mesh screen 213 may be a piezo-actuated vibrating mesh. For example, the mesh screen 213 may be coupled with a piezoelectric transducer and/or actuator, which may include a piezo crystal that oscillates when provided with an electronic signal of an appropriate excitation frequency. The elastic pouch 221 may be formed into a perimeter wall of the first reservoir 203 such that the elastic pouch 221 may prevent the first vaporizable material 211 from leaking out of the content chamber 209. The elastic pouch 221 may be formed of an elastic material and located opposite the mesh screen 213. In some example embodiments, the vaporizer body 201 may include a mechanical element 241 (e.g., an ultrasonic horn), which may be coupled to the first reservoir 203 for vaporizing the first vaporizable material 211.

FIG. 2B depicts a top view of an example of the mesh screen 213, in accordance with some example embodiments. As shown in FIG. 2B, the surface of the mesh screen 213 may include a plurality of apertures 259 through which a material, such as a vaporizable material, may be nebulized.

FIG. 2C depicts a perspective view of an example of the reservoir 203 including the mesh screen 213, in accordance with some example embodiments. As shown in FIG. 2C, the first reservoir 203 may include the content chamber 209, the first vaporizable material 211, and the mesh screen 213. In some example embodiments, the mesh screen 213 may be implemented and/or integrated into the first reservoir 203 such that the mesh screen 213 may form at least a portion of the first reservoir 203 such as, for example, one or more sides (e.g., bottom, top, sides) of the content chamber 209.

In some example embodiments, the controller 250 may activate a piezoelectric actuator 261 to vibrate the mesh screen 213 such that a portion of the first vaporizable material 211 may vaporize and/or nebulize through the apertures 259 of the mesh screen 213 to produce a first aerosol (e.g., aerosol droplets). For example, an alternating vibration of the mesh screen 213 may build up alternating pressure in the first vaporizable material 211 that is near the mesh screen 213. The resulting pressure may push the first vaporizable material 211 through the apertures 259 generating the first aerosol. In some example embodiments, a different mechanism may be used to vibrate the mesh screen 213 and vaporize the first vaporizable material 211. For example, instead of the piezoelectric actuator 261, the controller 250 may activate the mechanical element 241, which may vibrate the mesh screen 213 to vaporize the first vaporizable material 211 and produce the first aerosol 263.

The mechanical element 241 may be used instead of the piezoelectric actuator 261 at least because the mechanical element 241 may be implemented as a part of the vaporizer body 101 whereas the piezoelectric actuator 261 may be implemented as a part of the cartridge 103. Including the piezoelectric actuator 261 in the cartridge 103 may increase the cost and the environmental impact associated with the cartridge 103, which may be undesirable when the cartridge 103 is configured to be disposable. Contrastingly, including the mechanical element 241 in the vaporizer body 101 may be more cost effective at least because the vaporizer body 101 may be a durable component configured for repeated use. Including the mechanical element 241 in the vaporizer body 101 may also be more environmentally friendly because, unlike the vaporizer cartridge 103, the vaporizer body 101 is reusable and discarded much less frequently than the vaporizer cartridge 103.

In some example embodiments, the second reservoir 205 may include one or more content chambers 225 containing the second vaporizable material 227 (e.g., nicotine), which may be different from the first vaporizable material 211 in the first reservoir 203. The second reservoir 205 may include an air inlet 229, an outlet 231, and a vapor-air chamber 233. The second reservoir 205 may also include a heating element 235 (e.g., a coil, a convective heater) disposed at least partially around a wick 237 configured to draw, via a capillary action, at least a portion of the second vaporizable material 227 that is stored in the one or more content chambers 225. In some example embodiments, the heating element 235 may operate over a temperature range of approximately 120 degree-Celsius to 500 degree-Celsius. The heating element 235 may include one or more terminals 239. In some example embodiments, the vaporizer body 201 may include one or more electrical contact pads 243, an air inlet 245, an air outlet 247, a pressure sensor 249, a power source (e.g., a battery), and one or more controllers 250. In some example embodiments, the mouthpiece 207 may include one or more inlets 251 and 253, a mixing chamber 255, and an outlet 257.

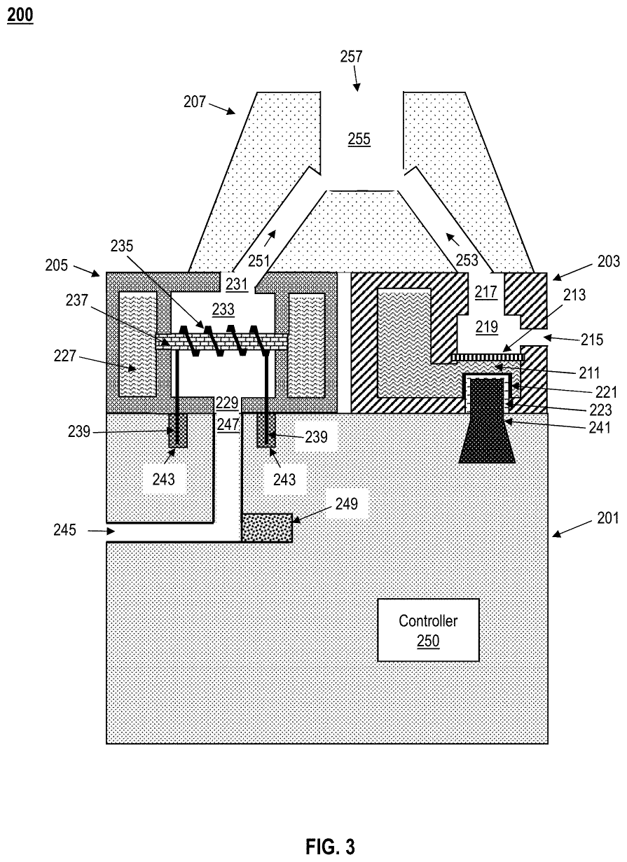

FIG. 3 depicts a block diagram illustrating another example of the vaporizer device 200, in accordance with some example embodiments. In some implementations of the current subject matter, a cartridge such as cartridge 103, may include multiple reservoirs, for example, the first reservoir 203 including the first vaporizable material 211 and the second reservoir 205 including the second vaporizable material 227. In the example of the vaporizer device 200 shown in FIG. 3, the first reservoir 203 may be coupled with the vaporizer body 201 such that a section of the mechanical element 241 may align with and enter the elastic pouch 221 expanding the elastic pouch 221 into the first vaporizable material 211. Although the portion of the mechanical element 241 may expand the elastic pouch 221, the elastic pouch 221 may remain unpunctured while preventing the liquid 223 and the first vaporizable material 211 from leaking out. In some example embodiments, the mechanical element 241 may generate ultrasonic vibrations, which the liquid 223 may transfer to the first vaporizable material 211. The transferred energy may vibrate the first vaporizable material 211 and/or the mesh screen 213 causing a portion of the first vaporizable material 211 to vaporize and/or nebulize through the apertures of the mesh screen 213 producing a first aerosol in the vapor-air chamber 219 of the first reservoir 203. In some example embodiments, the first vaporizable material 211 may be at or above ambient temperature while the portion of the first vaporizable material 211 is vaporized. In some example embodiments, the first aerosol in the vapor-air chamber 219 may exit an outlet 217, which may be coupled to the inlet 253 of the mouthpiece 207.

In some example embodiments, the second reservoir 205 may be coupled to the vaporizer body 201 such that each of the terminals 239 may be coupled to one of the electrical contacts 243. Upon a user drawing air through the outlet 257 of the mouthpiece 207, a pressure sensor 249 may detect a pressure change due to an airflow through the air inlet 245. Upon detecting the pressure change, the controller 250 may activate the electrical contacts 243 and the mechanical element 241. The electrical contacts 243 may energize the heating element 235 to heat the second vaporizable material 227 that has been absorbed by the wick 237.

In some example embodiments, the controller 250 may be configurable (e.g., by a user, an algorithm, an application, etc.) to control a vaporization rate at which each of the first vaporizable material 211 and the second vaporizable material 227 are vaporized to generate, respectively, the first aerosol and the second vapor. The vaporization rates for each of the first vaporizable material 211 and the second vaporizable material 227 may be different or same. Moreover, the vaporization rate for each of the first vaporizable material 211 and the second vaporizable material 227 may vary based on predefined periods. For example, the vaporization rate of the first vaporizable material 211 may be set at every 10 seconds whereas the vaporization rate of the second vaporizable material 227 may be set at every five seconds. Additionally and/or alternatively, the vaporization rate of the first vaporizable material 211 may be set for a duration of five milliseconds whereas the vaporization rate of the second vaporizable material 227 may be set for a duration of 10 milliseconds.

Moreover, the vaporization rate for each of the first vaporizable material 211 and the second vaporizable material 227 may be based on an airflow rate at the air inlets 215, 245, and/or 401. For example, a stronger or longer-lasting airflow at the air inlets 215, 245, and/or 401 may cause a longer duration of the vaporization for each the first vaporizable material 211 and the second vaporizable material 227, and the like. In some example embodiments, a user and/or the controller 250 may change one or more functional parameters of the mechanical element 241 and/or the piezoelectric actuator 261 to control the vaporization rate of the first vaporizable material 211. For example, the user and/or the controller 250 may change a vibrating amplitude (e.g., an amplitude of an AC signal), a vibrating frequency (e.g., a frequency of an AC signal), and/or a duty cycle of the mechanical element 241. Additionally or alternatively, the user and/or the controller 250 may change one or more parameters (e.g., frequency, amplitude, and/or the like) of an input signal to the mechanical element 241 to control the vibration of the first vaporizable material 211 that may affect the vaporization rate of the first vaporizable material 211.

In some example embodiments, a user and/or the controller 250 may change one or more functional parameters of the heating element 235 to control the vaporization rate of the second vaporizable material 227. For example, the user and/or the controller 250 may change the power level supplied to the heating element 235, change the target temperature of the heating element 235, and/or change the duty cycle of the power supply for the heating element 235.

In some example embodiments, the vaporizer body 201 and/or the reservoir 205 may include a heating mesh, a heating plate, a heating tube, and the like that may be utilized to heat the second vaporizable material 227 generating a second aerosol in the vapor-air chamber 233. Ambient air from the air inlet 245 may enter the vapor-air chamber 233 and force the second aerosol in the vapor-air chamber 233 to exit via the outlet 231 and enter the inlet 251 of the mouthpiece 207. In some example embodiments, upon detecting the pressure change, the controller 250 may also activate the mechanical element 241, which may generate an ultrasonic frequency forcing some of the first vaporizable material 211 against the mesh screen 213 causing vaporization of a portion of the first vaporizable material 211.

In some example embodiments, ambient air entering through the air inlet 215 may force the first aerosol in the vapor-air chamber 219 to exit via the outlet 217 and enter the inlet 253 of the mouthpiece 207. In some example embodiments, without or with minimal airflow from the air inlet 215, the piezoelectric vibration generated by the mechanical element 241 may cause the first aerosol in the vapor-air chamber 219 to exit via the outlet 217. The vapors from the first reservoir 203 and second reservoir 205 may enter the mouthpiece 207, via the inlets 251 and 253, and combine in the mixing chamber 255 before exiting the mouthpiece 207 via the outlet 257.

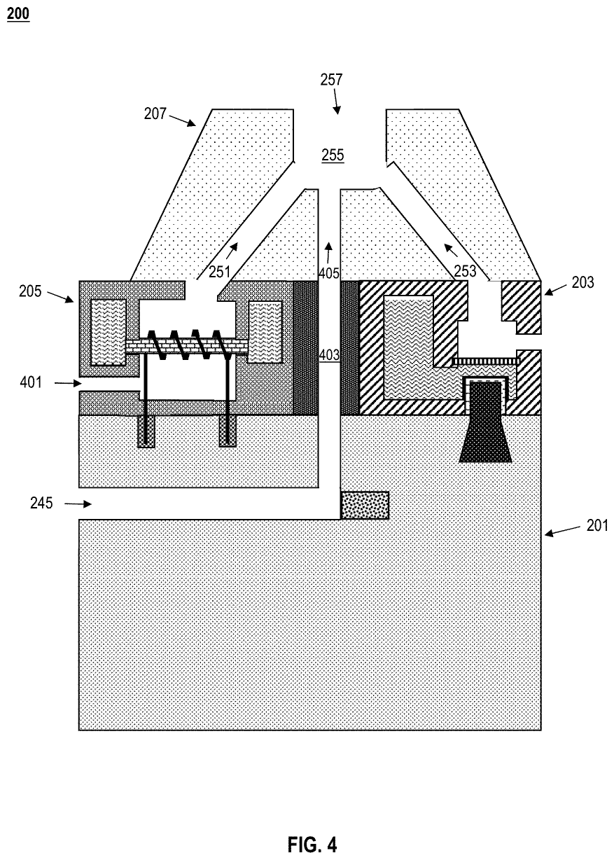

FIG. 4 depicts a block diagram illustrating another example of the vaporizer device 200, in accordance with some example embodiments. In the example of the vaporizer device 200 shown in FIG. 4, the second reservoir 205 may include an air inlet 401. The air inlet 245 may direct, to the mouthpiece 207, ambient air via a channel 403 located between the first reservoir 203 and second reservoir 205. Ambient air in the channel 403 may enter the mouthpiece 207 via an air inlet 405 while the vapors from the first reservoir 203 and second reservoir 205 may enter the mouthpiece 207 via the inlets 253 and 251, respectively.

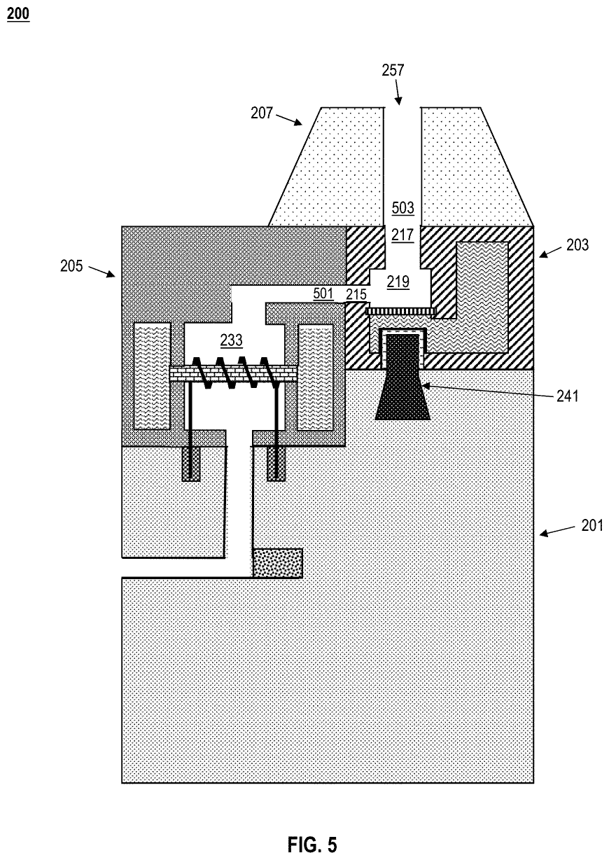

FIG. 5 depicts a block diagram illustrating another example of the vaporizer device 200, in accordance with some example embodiments. In some example embodiments, the mechanical element 241 and the first reservoir 203 may be configured such that the air inlet 215 may be coupled to an outlet 501 of the second reservoir 205. The second aerosol in the vapor-air chamber 233 may exit via the outlet 501 of the second reservoir 205, enter the first reservoir 203 via the air inlet 215, combine with the first aerosol in the vapor-air chamber 219 of the first reservoir 203, exit via the outlet 217 of the first reservoir 203, and enter an inlet 503 of the mouthpiece 207. In some example embodiments, the vapors from the first reservoir 203 and second reservoir 205 may combine in the vapor-air chamber 219 before entering the inlet 503 of the mouthpiece 207. In some example embodiments, the configuration of the first reservoir 203 and second reservoir 205 may be reversed such that ambient air from the air inlet 215 may enter the first reservoir 203 forcing the first aerosol into the chamber 233 of the second reservoir 205. A combined first aerosol and second aerosol may then enter the mouthpiece 207.

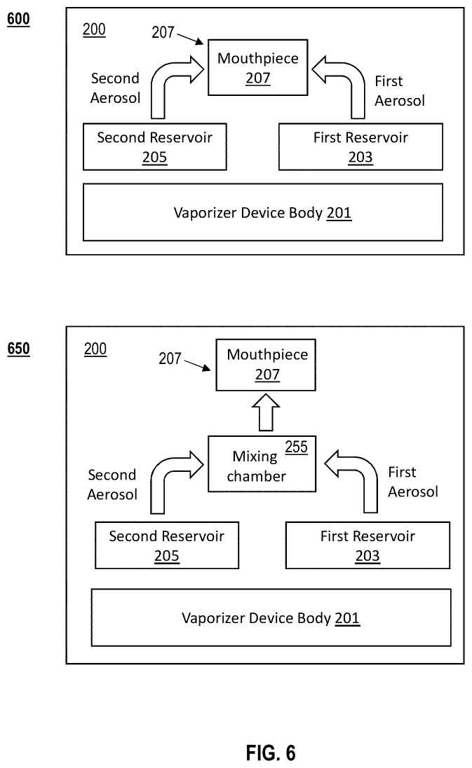

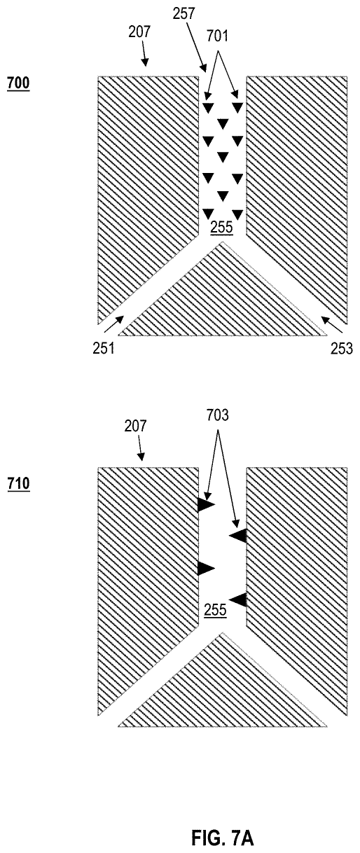

FIG. 6 depicts schematic diagrams 600 and 650 of examples of mechanisms for mixing aerosols in the vaporizer device 200, in accordance with some example embodiments. In the schematic diagram 600, the first aerosol generated by vaporizing the first vaporizable material 211 from the first reservoir 203 and the second aerosol generated by vaporizing the second vaporizable material 227 from the second reservoir 205 may be directed to a mouthpiece 207. In the absence of a mixing chamber, the first aerosol and the second aerosol are not subject to any mixing prior to being directed to the mouthpiece 207.

Alternatively, in the schematic diagram 650, the first aerosol generated by vaporizing the first vaporizable material 211 from the first reservoir 203 and the second aerosol generated by vaporizing the second vaporizable material 227 from the second reservoir 205 may be directed to a mixing chamber 255 such that the first aerosol and the second aerosol are combined before entering the mouthpiece 207. In some example embodiments, the mixing chamber 255 may be a standalone element or may be implemented as part of the mouthpiece 207 of the vaporizer device 200.

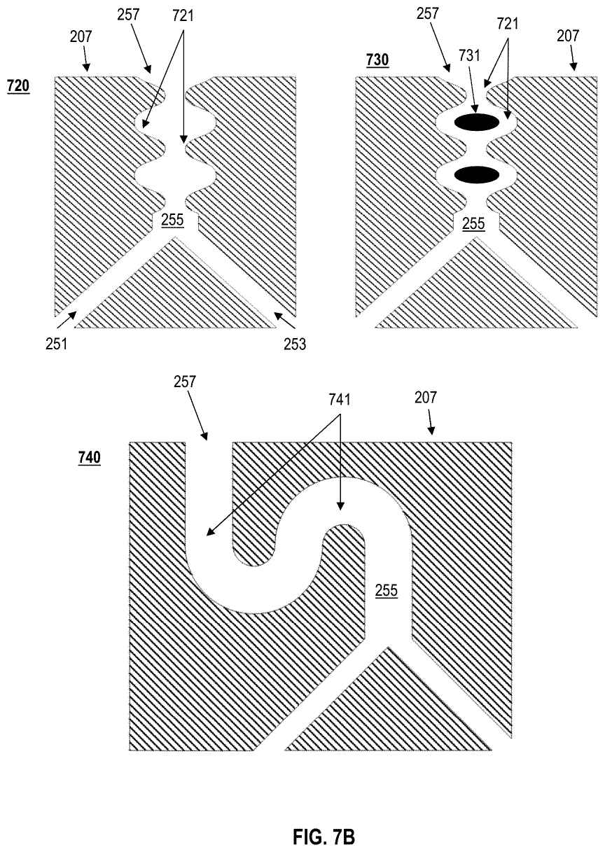

FIG. 7A depicts an example configuration of the mouthpiece 207 of the vaporizer device 200, in accordance with some example embodiments. As shown in diagram 700, the mouthpiece 207 may include the mixing chamber 255, which may include a plurality of obstacles 701. In some implementations, the obstacles 701 may alter flow rates and/or flow paths of the first and second vapors, which may enter the mixing chamber 255 via the inlets 251 and 253, such that the first and second vapors may combine together in the mixing chamber 255 before exiting the mouthpiece 207 via the outlet 257. Diagram 710 illustrates the mouthpiece 207 including the mixing chamber 255, which may include a plurality of obstacles 703, which may be of a different size and have different orientations compared to the obstacles 701. The obstacles 703 may alter flow rates and/or flow paths of the first and second vapors in the mixing chamber 255 such that the first and second vapors may combine together in the mixing chamber 255 before exiting the mouthpiece 207.

FIG. 7B depicts additional example configurations of the mouthpiece 207 of the vaporizer device 200, in accordance with some example embodiments. In some implementations, the mixing chamber 255 may include non-linear flow paths 721, which may alter flow rates and/or flow paths of the first and second vapors entering the mixing chamber 255 via the inlets 251 and 253. The non-linear flow paths 721 may cause a mixing and/or a combining of the first and second vapors in the mixing chamber 255 before a mixed/combined aerosol exits the mouthpiece 207 via the outlet 257. Also depicted is diagram 730 illustrating the mouthpiece 207 including the mixing chamber 255, which may include the non-linear flow paths 721 and one or more obstacles 731. In some implementations, the non-linear flow paths 721 and one or more obstacles 731 may alter flow rates and/or flow paths of the first and second vapors such that the first and second vapors may combine together in the mixing chamber 255 before exiting the mouthpiece 207 via the outlet 257. Diagram 740 illustrates the mouthpiece 207 including the mixing chamber 255, which may include a longer, non-linear flow path 741. In some implementations, the non-linear flow path 741 may alter flow rates and/or flow paths of the first and second vapors such that the first and second vapors may combine together in the mixing chamber 255 before exiting the mouthpiece 207 via the outlet 257.

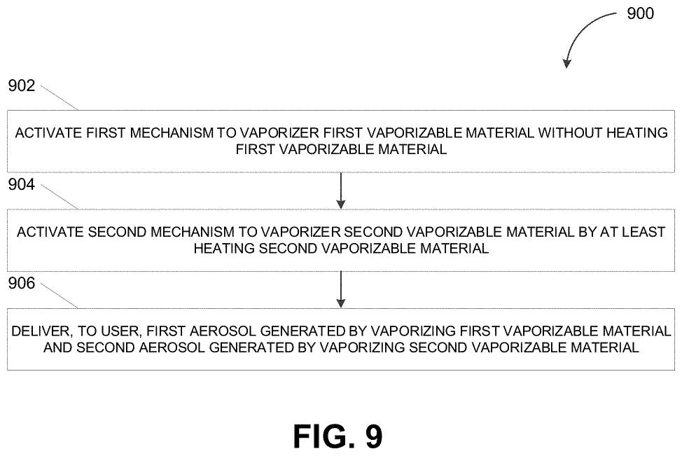

FIG. 9 depicts a flowchart illustrating an example of a process 900 for providing an aerosol, in accordance with some example embodiments. Referring to FIGS. 1-9, the process 900 may be performed by the vaporizer device 200.

At 902, a first mechanism is activated to vaporize a first vaporizable material without heating the first vaporizable material. For example, in some example embodiments, the vaporizer device 200 may include the first reservoir 203 holding the first vaporizable material 211, which may be vaporized by a first mechanism that does not heat the first vaporizable material 211 to change a temperature of the first vaporizable material 211. The first mechanism may include the piezoelectric actuator 261, which may vibrate the mesh screen 213 to vaporize the first vaporizable material 211 and generate the first aerosol. Alternatively and/or additionally, the first mechanism may include the mechanical element 241 configured to vibrate the mesh screen 213 to vaporize the first vaporizable material 211 and produce the first aerosol. As noted, the piezoelectric actuator 261 may be included in the cartridge 103 whereas the mechanical element 241 may be included in the vaporizer body 101.

At 904, a second mechanism is activated to vaporize a second vaporizable material by at least heating the second vaporizable material. In some example embodiments, the vaporizer device 200 may further include the second reservoir 205 holding the second vaporizable material 227, which may be vaporized by a second mechanism. The second mechanism may include the heating element 235, which may be configured heat the second vaporizable material 227 absorbed by the wick 237 to generate the second aerosol.

At 906, a first aerosol generated by vaporizing the first vaporizable material and a second aerosol generated by vaporizing the second vaporizable material are delivered to a user. In some example embodiments, the first aerosol generated by vaporizing the first vaporizable material 211 from the first reservoir 203 and the second aerosol generated by vaporizing the second vaporizable material 227 from the second reservoir 205 may be directed to a mouthpiece 207 for delivery to a user of the vaporizer device 200. The vaporizer device 200 may include the mixing chamber 255, in which case the first aerosol and the second aerosol are mixed before being delivered to the user. Alternatively, in the absence of the mixing chamber 255, the first aerosol and the second aerosol are directed to the mouthpiece 207 for delivery to the user without being subject to any mixing.

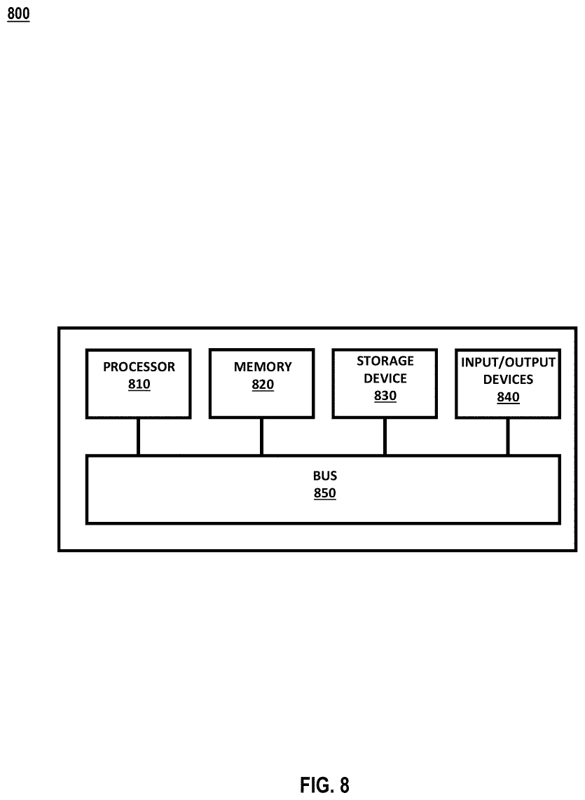

FIG. 8 depicts a block diagram illustrating a computing system 800 consistent with some implementations of the current subject matter. The computing system 800 may implement, for example, the controller 250 of the vaporizer device 200 and/or any components therein.

As shown in FIG. 8, the computing system 800 can include a processor 810, a memory 820, a storage device 830, and input/output devices 840. The processor 810, the memory 820, the storage device 830, and the input/output devices 840 can be interconnected via a system bus 850. The processor 810 is capable of processing instructions for execution within the computing system 800. Such executed instructions can implement one or more components of, for example, the controller 250. In some example embodiments, the processor 810 can be a single-threaded processor. Alternately, the processor 810 can be a multi-threaded processor. The processor 810 is capable of processing instructions stored in the memory 820 and/or on the storage device 830 to display graphical information for a user interface provided via the input/output device 840.

The memory 820 is a computer-readable medium such as volatile or non-volatile that stores information within the computing system 800. The memory 820 can store data structures representing configuration object databases, for example. The storage device 830 is capable of providing persistent storage for the computing system 800. The storage device 830 can be a floppy disk device, a hard disk device, a solid-state disk device, a flash drive device, an optical disk device, or a tape device, or other suitable persistent storage means. The input/output device 840 provides input/output operations for the computing system 800. In some example embodiments, the input/output device 840 includes a keyboard and/or pointing device. In various implementations, the input/output device 840 includes a display unit for displaying graphical user interfaces.

According to some example embodiments, the input/output device 840 can provide input/output operations for a network device. For example, the input/output device 840 can include Ethernet ports or other networking ports to communicate with one or more wired and/or wireless networks (e.g., a local area network (LAN), a wide area network (WAN), the Internet).

In some example embodiments, the computing system 800 can be used to execute various interactive computer software applications that can be used for implementing a local controller, a remote controller, and/or an inspection module. Alternatively, the computing system 800 can be used to execute any type of software applications. These applications can be used to perform various functionalities, e.g., reporting functionalities (e.g., generating, managing, editing of spreadsheet documents, word processing documents, and/or any other objects, etc.), computing functionalities, communications functionalities, etc. The applications can include various add-in functionalities (e.g., for manufacturing processes and/or another type of program) or can be standalone computing products and/or functionalities. Upon activation within the applications, the functionalities can be used to generate the user interface provided via the input/output device 840. The user interface can be generated and presented to a user by the computing system 800 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive trackpads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and sub-combinations of the disclosed features and/or combinations and sub-combinations of several further features disclosed above. In addition, the logic flows depicted in the accompanying figures and/or described herein do not necessarily require the particular order shown, or sequential order, to achieve desirable results. Other implementations may be within the scope of the following claims.

While this specification contains many specifics, these should not be construed as limitations on the scope of what may be claimed or of what may be claimed, but rather as descriptions of features specific to particular implementations or embodiments. Certain features that are described in this specification in the context of separate implementations may also be implemented in combination in a single implementation. Conversely, various features that are described in the context of a single implementation may also be implemented in multiple implementations or embodiments separately or in any suitable sub-combination. Moreover, although features may be described above as acting in certain combinations and even initially claimed as such, one or more features from a claimed combination may in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications, and enhancements to the described examples and implementations and other implementations may be made based on what may be disclosed.

1. A vaporizer device comprising:
a first reservoir containing or configured to contain a first vaporizable material that includes at least one first aerosol component;
a first aerosol generation mechanism configured to create a first part of an aerosol that includes the at least one first aerosol component, the first part of the aerosol being formed by a vibration generated by the first aerosol generation mechanism while the first vaporizable material is maintained at a first temperature below a first vaporization temperature of the at least one first aerosol component;
a second reservoir containing or configured to contain a second vaporizable material that includes at least one second aerosol component; and
a second aerosol generation mechanism configured to vaporize the second aerosol component of the second vaporizable material to generate a second part of the aerosol.

2. (canceled)

3. The vaporizer device of claim 1, wherein the vaporizer device comprises a vaporizer device body that is configured to be re-usable and to be joinable with at least one separable cartridge.

4. The vaporizer device of claim 3, wherein the at least one separable cartridge comprises the first reservoir, the second reservoir and at least a portion of each of the first aerosol generation mechanism and the second aerosol generation mechanism.

5. The vaporizer device of claim 3, wherein the at least one separable cartridge comprises one or more of the first reservoir, the second reservoir, a mouthpiece, and electrical connections for receiving power from a battery or other power source in the vaporizer device body when the vaporizer device body and cartridge are coupled.

6. The vaporizer device of claim 1, further comprising:
a pressure sensor configured to detect a pressure change corresponding to an airflow through an air inlet; and
a controller configured to respond to the pressure change by at least activating the first aerosol generation mechanism and/or the second aerosol generation mechanism.

7. The vaporizer device of claim 6, wherein the controller is further configured to control a first vaporization rate of the first aerosol component by at least adjusting an amplitude, a frequency, and/or a duty cycle of the vibration associated with the first aerosol generation mechanism.

8. The vaporizer device of claim 6, wherein the controller is further configured to control a second vaporization rate of the second aerosol component by at least adjusting a power level, a duty cycle of a power supply, and/or a target temperature associated with the second aerosol generation mechanism.

9. The vaporizer device of claim 1, wherein the first temperature is an ambient temperature.

10. The vaporizer device of claim 1, wherein the first temperature is lower than the second temperature.

11. The vaporizer device of claim 1, wherein the second aerosol generation mechanism includes a heating element, and wherein the heating element is configured to generate heat for heating the second aerosol component to the second temperature in order to vaporize the second aerosol component.

12. The vaporizer device of claim 1, wherein the first aerosol generation mechanism includes a piezoelectric actuator configured to generate an ultrasonic vibration, and wherein the ultrasonic vibration vibrates a mesh screen to vaporize the first aerosol component without generating heat to change the first temperature of the first aerosol component.

13. The vaporizer device of claim 12, wherein the piezoelectric actuator is included in a first vaporizer cartridge.

14. The vaporizer device of claim 1, wherein the first aerosol generation mechanism includes a mechanical horn configured generate an ultrasonic vibration that vibrates a mesh screen to vaporize the first aerosol component without generating heat to change the first temperature of the first aerosol component.

15. (canceled)

16. The vaporizer device of claim 1, further comprising:
a mouthpiece configured to deliver, to a user, the first part of the aerosol and the second part of the-aerosol, the mouthpiece including an aerosol outlet through which the first part of the aerosol and the second part of the aerosol exit the mouthpiece.

17. The vaporizer device of claim 16, wherein the first part of the aerosol enters the mouthpiece through a first inlet in the mouthpiece and the second part of the aerosol enters the mouthpiece through a second inlet of the mouthpiece such that the first part of the aerosol and the second part of the aerosol enter the mouthpiece without any mixing.

18. The vaporizer device of claim 16, wherein the first part of the aerosol and the second part of the aerosol are mixed prior to entering the mouthpiece.

19. The vaporizer device of claim 18, wherein the mouthpiece is in fluid communication with a mixing chamber having one or more features configured to combine the first part of the aerosol with the second part of the aerosol.

20. The vaporizer device of claim 19, wherein the one or more features include a non-linear flow path and/or an obstacle.

21. A method, comprising:
activating a first aerosol generation mechanism to vaporize a first vaporizable material included in a first reservoir, the first aerosol generation mechanism configured to generate a vibration to vaporize the first vaporizable material while the first vaporizable material is maintained at a first temperature below a first vaporization temperature of at least one first aerosol component comprising the first vaporizable material;
activating a second aerosol generation mechanism to vaporize a second vaporizable material included in a second reservoir, the second mechanism configured to vaporize the second vaporizable material at a second temperature near or above a second vaporization temperature of at least one second aerosol component comprising the second vaporizable material; and
delivering, to a user, a first aerosol generated by vaporizing the first vaporizable material and a second aerosol generated by vaporizing the second vaporizable material.

22-34. (canceled)

35. A vaporizer device, comprising:
a first reservoir configured to contain a first vaporizable material that includes at least one first aerosol component;
a first aerosol generation mechanism configured to generate a vibration to vaporize the at least one first aerosol component to generate a first part of an aerosol, the vibration forming the first aerosol component without causing delivery of significant amounts of heat to the first vaporizable material and/or to the first aerosol component;
a second reservoir configured to contain a second vaporizable material that includes at least one second aerosol component; and
a second aerosol generation mechanism configured to vaporize at least one second aerosol component of the second vaporizable material to generate a second part of the aerosol, the second aerosol generation mechanism configured to vaporize, at a second temperature near or above a vaporization temperature of the at least one second aerosol component, the at least one second aerosol component to generate the second part of the aerosol.
